(19) United States
(12) Patent Application Publication
(10) Pub. No.: US 2014/0221822 A1
(43) Pub. Date: Aug. 7, 2014

(54) INSTRUMENT DEPTH TRACKING FOR OCT-GUIDED PROCEDURES

(71) Applicant: THE CLEVELAND CLINIC FOUNDATION, Cleveland, OH (US)

(72) Inventors: Justis P. Ehlers, Shaker Hts., OH (US); Sunil K. Srivastava, Shaker Hts., OH (US); Yuankai Tao, Shaker Hts., OH (US)

(73) Assignee: THE CLEVELAND CLINIC FOUNDATION, Cleveland, OH (US)

(21) Appl. No.: 14/172,424

(22) Filed: Feb. 4, 2014

Related U.S. Application Data
(60) Provisional application No. 61/760,357, filed on Feb. 4, 2013.

(51) Int. Cl. A61B 5/06

(52) U.S. Cl. CPC A61B 5/061
USPC 600/424

(57) ABSTRACT

Systems and methods are provided for tracking a depth of a surgical instrument in an optical coherence tomography (OCT) guided surgical procedure. An OCT device is configured to image a region of interest to provide OCT data. A scan processor is configured to determine a relative position of the instrument and a target within the region of interest from at least the OCT data, where the instrument is one of in front of the target, within the target, or below the target. A feedback element is configured to communicate the relative position of the instrument and the target to a user in a human comprehensible form.

### RELATED APPLICATION

This application claims priority from U.S. Provisional Application No. 61/760,357, filed 4 Feb. 2013, the subject matter of which is incorporated herein by reference in its entirety.

### TECHNICAL FIELD

The present invention relates generally to the field of medical devices, and more particularly to systems and methods for tracking the depth of an instrument in an optical coherence tomography (OCT) guided procedure.

### BACKGROUND OF THE INVENTION

Over the years, multiple milestones have revolutionized ophthalmic surgery. X-Y surgical microscope control, wide-angle viewing, and fiberoptic illumination are all examples of instrumentation that have been integrated to radically improve pars plana ophthalmic surgery. Optical coherence tomography (OCT) has dramatically increased the efficacy of treatment of ophthalmic disease through improvement in diagnosis, understanding of pathophysiology, and monitoring of progression over time. Its ability to provide a high-resolution, cross-sectional, three-dimensional view of the relationships of ophthalmic anatomy during surgery makes intraoperative OCT a logical complement to the ophthalmic surgeon.

### SUMMARY OF THE INVENTION

In accordance with an aspect of the prevent invention, a system is provided for tracking a depth of a surgical instrument in an optical coherence tomography (OCT) guided surgical procedure. An OCT device is configured to image a region of interest to provide OCT data. A scan processor is configured to determine a relative position of the instrument and a target within the region of interest from at least the OCT data, where the instrument is one of in front of the target, within the target, or below the target. A feedback element is configured to communicate the relative position of the instrument and the target to a user in a human comprehensible form.

In accordance with another aspect of the invention, a computer-implemented method is provided for communicating a relative location of a surgical instrument and a target within a region of interest to a surgeon. An optical coherence tomography scan is performed of the region of interest to produce at least one set of A-scan data. An axial location of the surgical instrument and an axial location of the target are identified from the at least one set of A-scan data. A relative distance is calculated between the surgical instrument and the target, and the calculated relative distance between the surgical instrument and the target is communicated to the surgeon via one of a visual, a tactile, and an auditory feedback element. Each of identifying the axial location of the surgical instrument, identifying the axial location of the target, calculating the relative distance, and communicating the calculated relative distance are performed in real time, such that a change in the calculated relative distance is communicated to the surgeon after a sufficiently small interval as to be perceived as immediately responsive to a movement of the instrument.

In accordance with yet another aspect of the invention, a system is provided for tracking a surgical instrument in an optical coherence tomography (OCT) guided surgical procedure. An OCT device is configured to image a region of interest to provide OCT data. A scan processor is configured to determine an axial position of the surgical instrument and an axial position of a target within the region of interest from the OCT data. The scan processor includes a pattern recognition classifier to identify at least one of the instrument and the target. A feedback element is configured to communicate at least a relative position of the instrument and the target to a user in a human comprehensible form.

### BRIEF DESCRIPTION OF THE DRAWINGS

The foregoing and other features of the present invention will become apparent to those skilled in the art to which the present invention relates upon reading the following description with reference to the accompanying drawings, in which:

FIG. 1 illustrates one example of a system for tracking the depth of a surgical instrument in an OCT-guided surgical procedure in accordance with an aspect of the present invention;

FIG. 2 illustrates examples of displays of relative depth information in accordance with an aspect of the present invention.

FIG. 3 illustrates a first example of a surgical instrument, specifically an ophthalmic pic, optimized for use with the present invention as well as for optical coherence tomography;

FIG. 4 provides a close-up view of a working assembly associated with the ophthalmic pic;

FIG. 5 illustrates an OCT scan of a region of tissue with the ophthalmic pic of FIGS. 3 and 4 interposed between the OCT scanner and the tissue;

FIG. 6 illustrates a second example of a surgical instrument, specifically ophthalmic forceps, optimized for use with the present invention as well as for optical coherence tomography generally;

FIG. 7 provides a close-up view of a working assembly associated with the ophthalmic forceps;

FIG. 8 illustrates an OCT scan of a region of tissue with the ophthalmic forceps of FIGS. 6 and 7 interposed between the OCT scanner and the tissue; and

FIG. 9 illustrates a method for communicating a relative location of a surgical instrument and a target within a region of interest to a surgeon in accordance with an aspect of the present invention;

FIG. 10 illustrates an OCT scan of a region of tissue with an ophthalmic scraper with both the tissue and instrument segmented and the relative distances between the instrument and tissue layer of interest overlaid onto the OCT scan as a colormap for real-time surgical feedback; and

FIG. 11 is a schematic block diagram illustrating an exemplary system 200 of hardware components capable of implementing examples of the systems and methods disclosed herein, such as the instrument tracking system described previously.

### DETAILED DESCRIPTION

Optical Coherence Tomography (OCT) is a non-contact imaging modality that provides high resolution cross-sectional images of tissues of interest, including the eye and its microstructure. The ability to quickly image ophthalmic anatomy as a “light biopsy” has revolutionized ophthalmology. OCT is the most commonly performed imaging procedure in ophthalmology. The cross-sectional information provided by OCT is a natural complement to the ophthalmic surgeon. Real-time information could improve surgical precision, reduce surgical times, expand surgical capabilities, and improve outcomes.

Intraocular surgeries (e.g., cataract, corneal, vitreoretinal) could be impacted tremendously by the availability of intraoperative OCT. In cataract surgery, OCT-guided corneal incisions could improve wound construction, reducing hypotony and infection rates, as well as confirmation of anatomic location of intraocular lens insertions. In corneal surgery, intraoperative OCT would provide critical information in lamellar surgeries on graft adherence and lamellar architecture. For vitreoretinal surgery, OCT-assisted surgery will be critical to guiding membrane peeling in diabetic retinal detachments, macular puckers, and macular holes. Utilizing the methodologies described herein, real-time scanning could be performed to confirm the correct anatomic localization of instruments relative to structures of interest (e.g., vessel cannulation, intraocular biopsy, and specific tissue layer), provide rapid feedback to the surgeon regarding instrument location, identify key surgical planes, and provide depth information regarding the instrument's location within a tissue, above a tissue, or below a tissue.

One of the outstanding features of OCT is the high-resolution information that is gained from the A-scan that is subsequently summed for the cross-sectional view of the B-scan. The A-scan provides various peaks of reflectivity that are processed by the device. The various peaks and valleys of reflectivity on the A-scan and the summation of these peaks and valleys are exploited herein to “segment” the signal and provide depth and proximity information within the scan. The axial resolution is outstanding (e.g., 2-6 microns) in current SD-OCT systems.

The application of these technologies may be far reaching. OCT technology is now touching numerous fields throughout medicine (e.g., cardiology, dermatology, and gastroenterology). Diagnostic and surgical procedures are using OCT as an adjunct. Application of this invention to new devices within other specialties could broaden the diagnostic and therapeutic utility of OCT across medicine. Accordingly, properly optimized materials could also be utilized to create devices and instruments to be utilized in other areas of medicine which are already using OCT as a diagnostic modality but do not have instrumentation that is compatible with OCT to use it as a real-time adjunct to therapeutic maneuvers.

To this end, this invention provides a critical component for the integration of

OCT into surgical care. The systems and method described herein provide real-time processing of OCT signals during surgery, such that relative proximity information of an instrument and an anatomical structure can be extracted from an OCT scan and communicated to the surgeon. Specifically, when an instrument is introduced into the surgical field, it provides a specific reflection for the laser of OCT. This information, along with the tissue reflection, is processed by the OCT scanner to create an image. In accordance with an aspect of the present invention, either or both of hardware processing of the signals or software analysis of the reflectivity profile is utilized to provide the surgeon with rapid feedback of instrument location relative to the tissue, in effect “a depth gauge”. This system could be used with current instrumentation or OCT-optimized (i.e., OCT-friendly) instrumentation, described in detail below, that provides a more favorable reflectivity profile for visualizing underlying tissues. The feedback interface to the surgeon can be packaged in multiple formats to provide an individualized approach both to the needs of the surgical procedure as well as the desires of the surgeon

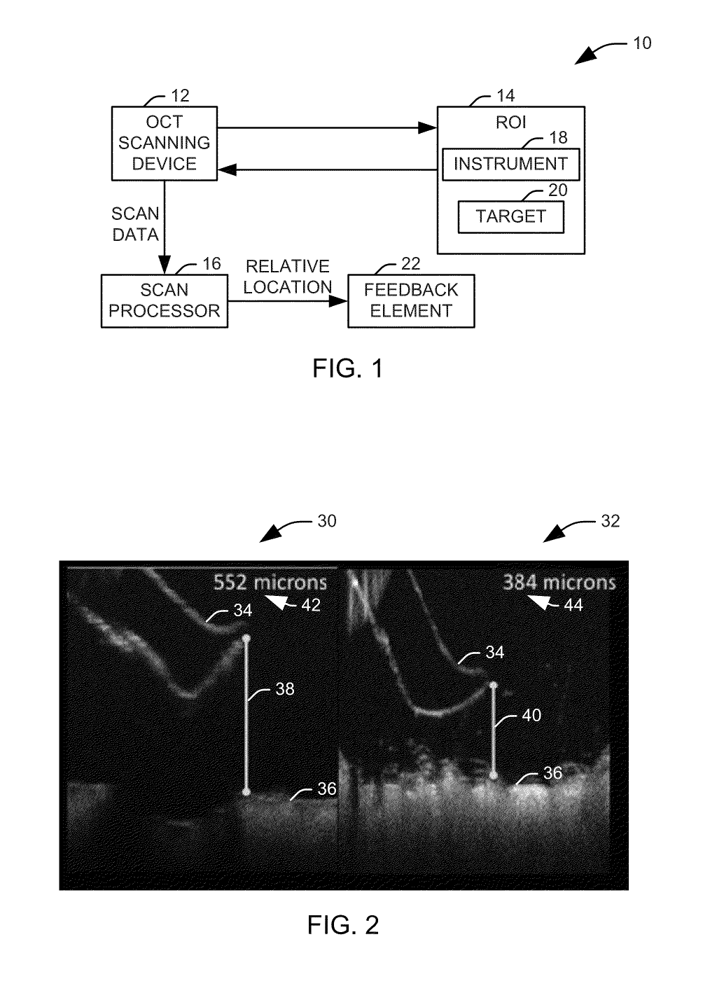

FIG. 1 illustrates one example of a system 10 for tracking the depth of a surgical instrument in an OCT-guided surgical procedure in accordance with an aspect of the present invention. The system 10 includes an OCT scanning device 12 configured to image a region of interest (ROI) 14 axially, that is, in a direction substantially parallel to a direction of emission of light from the OCT scanner. Specifically, for a given scan point, depending on the type of scanner, the OCT scanner can provide an axial reflectivity profile, referred to as an A-scan with very high resolution (e.g., on the order of several microns). Multiple such reflectivity profiles can be combined into a cross-sectional tomograph, referred to herein as a B-scan. It will be appreciated that various OCT scanning schemes utilize parallel or two-dimensional arrays to provide a cross-sectional or full-field tomography directly. For the purposes of this document, the term “A-scan” will be used to refer to an axial reflectivity profile representing a single, axially-aligned line segment.

A scan processor 16 receives the OCT data from the OCT scanning device 12 and determines a relative position of an instrument 18 and a target 20 within the region of interest 14. The target 20 can comprise a specific anatomical structure, a tissue surface, or any other landmark identifiable in an OCT scan. It will be appreciated that the scan processor 16 can be implemented as dedicated hardware, software or firmware instructions stored on a non-transitory computer readable medium and executed by an associated processor, or a combination of software and dedicated hardware.

In one implementation, the scan processor 16 can utilize known properties of the surgical instrument 18 to locate the instrument within raw A-scan data. For example, metallic portions of an instrument are highly reflective and effectively opaque to infrared light. Accordingly, an A-scan or set of A-scans showing a spike of returned light intensity above a threshold intensity at a given depth can be considered to represent the depth of the instrument. While the presence of a metallic instrument might obscure the underlying tissue, one or more adjacent A-scans could be utilized to determine an appropriate depth for the target 20, and a relative distance between the instrument 18 and the target 20 can be determined. OCT-friendly instruments, developed by the inventors and described in further detail below, might provide a reflection with significantly less intensity. In one example, a surface of the imaged tissue can be determined from the aggregate scan data, and reflections at depths above the determined surface can be determined to be the instrument 18.

In yet another implementation, the instrument 18 and the target 20 can be identified in cross-sectional or full-field tomography images via an appropriate pattern recognition algorithm. Given that this recognition would need to take place in near-real time to provide assistance to a surgeon during a medical procedure, these algorithms would likely exploit known properties of both the target 20 and the instrument 18 to maintain real-time processing. For example, the target 20 could be located during preparation for a surgery, and a relative position of the target 20 and one or more easily located landmarks could be utilized to facilitate location of the target. The instrument 18 can be located via a windowing operation that searches for non-uniform regions within the tissue. These regions can be segmented, with the segmented regions provided to a pattern recognition algorithm trained on sample images of the instrument 18 in or above tissue, as well as samples in which the instrument is not present, to confirm the presence of the instrument. Appropriate pattern recognition algorithms could include support vector machines, regression models, neural networks, statistical rule-based classifiers, or any other appropriate regression or classification model.

In one implementation, the instrument 18 can have one or more known profiles, representing, for example, different orientations of the instrument, and a template matching algorithm could be used to recognize the instrument within image data. To facilitate the template matching, the instrument 18 could be provided with one or more orientation sensors (e.g., an accelerometer, gyroscopic arrangement, magnetic sensor, etc.), and an appropriate template could be selected from a plurality of available templates according to the determined orientation relative to the OCT scanner 12.

Once a relative position of the instrument 18 and the target 20 has been determined, the relative position is communicated to the surgeon via a feedback element 22. It will be appreciated that the feedback element 22 can include any of one or more speakers to provide an audible indication to the surgeon, one or more displays to provide, for example, a numerical or graphical indicator, or one of more other visual indicators, such as a change in the hue or brightness of a light source, or a number of individual indicators active within an array of indicators, such as set of light emitting diodes. Options for the feedback interface can include, for example, direct visualization of the cross-section of the instrument and tissue of interest, variable audio feedback based on proximity and depth (e.g., an audio alert based on relative proximity), or numeric feedback within the operating microscope or an adjacent monitor revealing relative depth information.

In one implementation, the feedback element 22 is implemented to provide the relative position of the instrument 18 and the target 20 as a numerical value. Specifically, the surgeon can be provided with immediate information regarding the distance of the instrument 18 to the target 20, such as a tissue of interest that is visualized within the microscope, via a heads-up display system or external monitor system, or integrated into a surgical system such as the vitrectomy machine user interface system. Options for the display system include a direct label in the region of interest 14 and proximity gauge away from the actual B-scan image. In another implementation, the feedback element 22 can be implemented to communicate the proximity of the instrument 18 and the target 20 via a variable audio feedback to eliminate potential visual distraction of visual feedback. For example, the audio feedback can vary in one or more of pitch, volume, rhythm (e.g., a frequency with which individual tones are presented), tone length, and timbre based on instrument/tissue proximity.

The system 10 can utilize the OCT scan data to discriminate the relative proximity of an instrument to the tissue of interest (e.g., forceps above the retinal surface), or the relative depth of an instrument within a tissue of interest (e.g., locating a subretinal needle within the subretinal space, identifying the depth of an instrument within the retina, locating a needle at a specific depth level within the cornea). This allows for direct surgeon-feedback on instrument/tissue proximity and tissue/depth information. In microsurgical procedures, the en face view from the surgical microscope provides the surgeon with some depth information from both direct and indirect cues, but this depth information is not optimal.

Utilizing this system 10, the surgeon has quantitative feedback on the proximity of an instrument to the tissue—a tremendous advance in precision and safety. This also may provide an important advance for translating into robotic assisted surgery through providing a non-human source of proximity information. Intraoperative OCT continues to be an area of active research and is not currently being utilized in mainstream clinical care. The introduction of an instrument-tissue proximity feedback system would be a tremendous advance for image-guided surgery. In addition, providing intra-tissue depth information opens the door to tremendous advances in surgical precision for anatomic localization, such as for targeted drug delivery (e.g., outer retina gene therapy, subretinal drug delivery), needle placement for lamellar keratoplasty (e.g., DALK), and implant placement (e.g., INTACS). Other potential clinical applications of this technology could include active tracking of needle depth in deep anterior lamellar keratoplasty (corneal surgery) to localize needle prior to initiating injection, identification of a depth of peel for DMEK and DSAEK for stripping of endothelium and Descemet's membrane in corneal surgery, proper location for channel placement and implant placement for INTACS, providing appropriate depth gauge for limbal relaxing incisions and cataract wound incisions for cataract surgery, depth of dissection determination for glaucoma filtering surgeries and for verification of proper drainage device location, verification of instrument/tissue proximity for vitreoretinal surgeries, such as membrane peeling with forceps, scissors, surgical pic, vitrector, intraretinal depth determination for targeted intraretinal delivery of therapeutics (e.g., proteins, gene therapy), choroidal and suprachoroidal depth determination for optimal instrument localization and potential therapeutic delivery, subretinal depth localization for therapeutic delivery, device delivery, or surgical manipulation feedback, and preretinal localization and proximity for optimal instrument/tissue spacing for therapeutics or drug delivery (e.g., radiotherapy, application of stains/dyes).

In one implementation, the target 20 can be located from the OCT data, while the instrument 18 is detected through other means. For example, the system 10 can include an additional sensor (not shown) to identify the location of the instrument via spectroscopy, scattered light from the OCT device, or any other contrast mechanism that facilitates identification of the instrument 18. It will be appreciated that the sensor can track radiation sources that are different from that associated with the OCT device. For example, depth tracking can be done using spectroscopic detection of specific radiation sources attached to the surgical instrument 18, with the wavelength of the radiation source selected to be detectable at the sensor. Using a series of calibration steps, the extra-ocular space may be mapped to the retinal or cornea space for real-time tracking of the instrument. This can be accomplished, for example, using one or a combination of imaging using fluorescence and pattern recognition. In another implementation, an optical marker is attached to each instrument, and the markers are identified in the OCT data to track real-time surgical motion. Tracking of posterior tips of instruments may utilize computational calibration and scaling to match external motions with intraocular motions.

FIG. 2 illustrates two examples of displays 30 and 32 of relative depth information in accordance with an aspect of the present invention. The displays 30 and 32 each include an OCT image of an instrument 34 and a tissue surface 36. For each display, a respective graphical indication 38 and 40 is provided to emphasize the relative distance between the instrument 34 and the surface 36. In the illustrated implementation, the graphical indication 38 and 40 is a bright colored line extending from a tip of the instrument axially to the tissue surface 36. To supplement this graphical indication, each display 30 and 32 also includes a numerical indicator 42 and 44 of the distance in microns between the instrument 34 and the surface 36. Accordingly, a surgeon can determine at a glance the position of the instrument 34 relative to the tissue and proceed accordingly.

The inventors have found a major limiting factor for the use of OCT in the operating room is the lack of “OCT-friendly” instrumentation. Current materials and instruments are less suitable for OCT imaging due to blockage of light transmission and suboptimal reflectivity profiles limiting visualization of the instrument, underlying tissues, and instrument/tissue interactions. For example, metallic instruments exhibit absolute shadowing of underlying tissues due to a lack of light transmission. Additionally, the low light scattering properties of metal result in a pinpoint reflection that does not allow for the instrument to be visualized easily on OCT scanning. Silicone based materials have more optimal OCT reflectivity properties, however, silicone does not provide the material qualities to create the wide-ranging instrument portfolio needed for intraocular surgery (e.g., forceps, scissors, blades).

Accordingly, in accordance with the present invention, the depth finding system can be utilized with instruments designed to have optical properties to optimize visualization of underlying tissues while maintaining instrument visualization on the OCT scan. The unique material composition and design of these instruments maintains the surgical precision for microsurgical manipulations, while providing optimal optical characteristics that allow for intraoperative OCT imaging. The optical features of these materials include a high rate of light transmission to reduce the shadowing of underlying tissue. This allows tissues below the instruments to be visualized on the OCT scans while the instrument hovers above the tissue or approaches the tissue. Simultaneously, the materials can either have light scattering properties that are high enough to allow for visualization of the instrument contours and features on OCT imaging or be surfaced appropriately to provide these properties. Exemplary instruments can include intraocular ophthalmic forceps, an ophthalmic pic, curved horizontal scissors, keratome blades, vitrectors, corneal needles (e.g., DALK needles), and subretinal needles, although it will be appreciated that other devices are envisioned.

In these instruments, the working assembly can be designed such that it does not significantly interfere with the transmission of infrared light between the eye tissue and the OCT sensor. Specifically, the working assembly can be formed from a material having appropriate optical and mechanical properties. In practice, the working assembly is formed from materials that are optically clear (e.g., translucent or transparent) at a wavelength of interest and have a physical composition (e.g., tensile strength and rigidity) suitable to the durability and precision need of surgical microinstruments. Exemplary materials include but are not limited to polyvinyl chloride, glycol modified poly(ethylene terephthalate) (PET-G), poly(methyl methacrylate) (PMMA), and polycarbonate.

In one implementation, the material of the working assembly is selected to have an index of refraction, for the wavelength of light associated with the OCT scanner, within a range close to the index of refraction of the eye tissue media (e.g., aqueous, vitreous). This minimizes both reflection of the light from the instrument and distortion (e.g., due to refraction) of the light as it passes through the instrument. In one implementation, the index of refraction of the material is selected to be between 1.3 and 1.6. The material is also selected to have an attenuation coefficient within a desired range, such that tissue underneath the instrument is still visible. Since attenuation is a function of the thickness of the material, the attenuation coefficient of the material used may vary with the specific instrument or the design of the instrument.

Looking at two examples, polycarbonate has excellent transmittance of infrared light, and an index of refraction in the near infrared band (e.g., 0.75-1.4 microns) just less than 1.6. It has a tensile modulus of around 2400 MPa. PMMA has varied transmittance across the near infrared band, but has minimal absorption in and around the wavelengths typically associated with OCT scanning. PMMA has an index of refraction in the near infrared band of around 1.48, and a tensile modulus between 2200 and 3200 MPa.

The inventors have determined that several materials with otherwise desirable properties provide insufficient diffuse reflectivity for a desired clarity of visualization of the instrument during an OCT scan. For example, certain transparent plastics have an amorphous microscopic structure and do not provide a high degree of diffuse scattering in the infrared band. In accordance with another aspect of the present invention, a surface of the working assembly can be abraded or otherwise altered in texture to provide a desired degree of scattering, such that the instrument is visible in the OCT scan without shadowing the underlying tissue. In one implementation, this shading is limited to the contact surface to provide maximum clarity of the tissue within the scan, but it will be appreciated that, in many applications, it will be desirable to provide surface texturing to the entirety of the surface of the working assembly to allow for superior visibility of the instrument, and thus increases accuracy of localization.

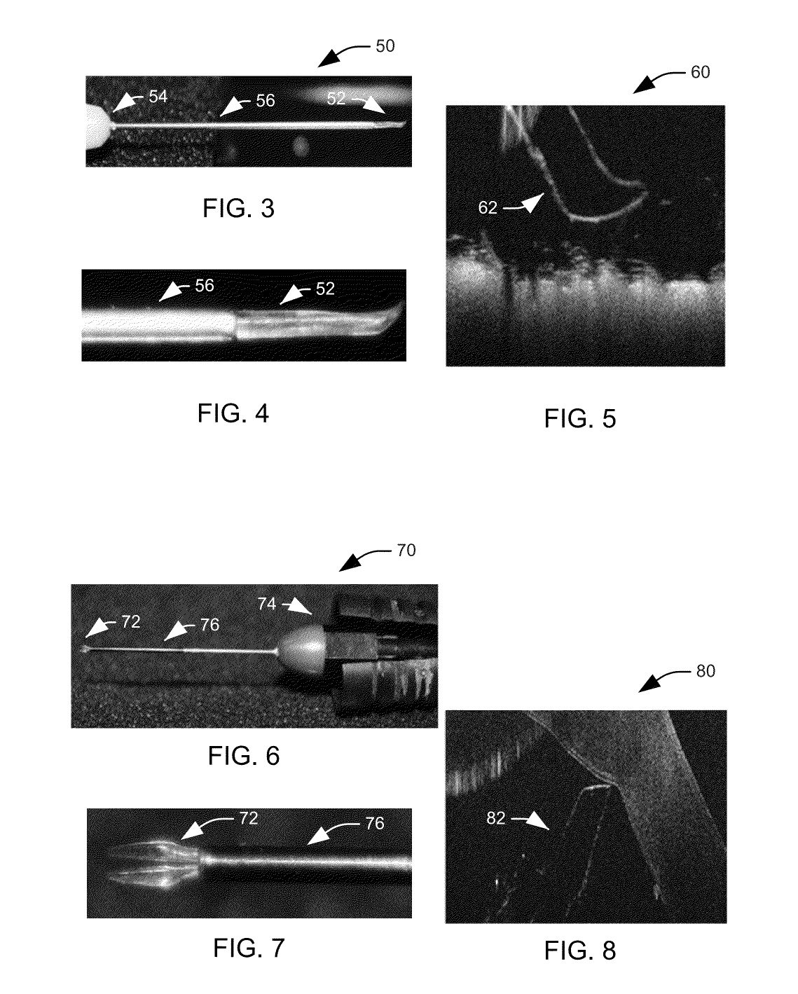

FIG. 3 illustrates a first example of a surgical instrument 50, specifically an ophthalmic pic, in accordance with an aspect of the present invention. FIG. 4 provides a close-up view of a working assembly 52 associated with the instrument 50. The instrument 50 has a handle 54 configured to be easily held by a user and a shaft 56 connecting the working assembly 52 to the handle. The working assembly 52 formed from polycarbonate and can, optionally, have surfacing applied to increase the diffuse reflection provided by the polycarbonate. FIG. 5 illustrates an OCT scan 60 of a region of eye tissue with the ophthalmic pic 50 of FIGS. 3 and 4 interposed between the OCT scanner and the tissue. A shadow 62 of the instrument is visible in the OCT scan 60, but it will be noted that the tissue under the instrument remains substantially visible.

FIG. 6 illustrates a second example of a surgical instrument 70, specifically ophthalmic forceps, in accordance with an aspect of the present invention. FIG. 7 provides a close-up view of a working assembly 72 associated with the instrument 70. The instrument 70 has a handle 74 configured to be easily held by a user and a shaft 76 connecting the working assembly 72 to the handle. The working assembly 72 formed from polycarbonate and can, optionally, have surfacing applied to increase the diffuse reflection provided by the polycarbonate. FIG. 8 illustrates an OCT scan 80 of a region of eye tissue with the ophthalmic forceps 70 of FIGS. 6 and 7 interposed between the OCT scanner and the tissue. Again, a shadow 82 of the instrument is visible in the OCT scan 80, but it will be noted that the tissue under the instrument remains substantially visible.

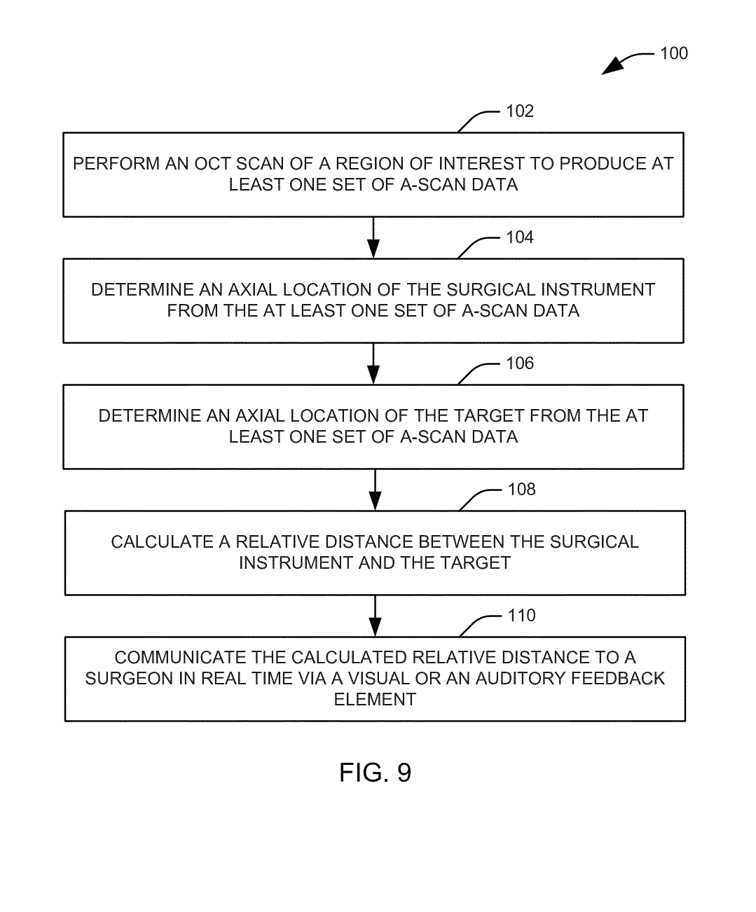

In view of the foregoing structural and functional features described above, methodologies in accordance with various aspects of the present invention will be better appreciated with reference to FIG. 9. While, for purposes of simplicity of explanation, the methodology of FIG. 9 is shown and described as executing serially, it is to be understood and appreciated that the present invention is not limited by the illustrated order, as some aspects could, in accordance with the present invention, occur in different orders and/or concurrently with other aspects from that shown and described herein. Moreover, not all illustrated features may be required to implement a methodology in accordance with an aspect the present invention.

FIG. 9 illustrates a method 100 for communicating a relative location of a surgical instrument and a target within a region of interest to a surgeon in accordance with an aspect of the present invention. It will be appreciated that the method 100 can be performed via either dedicated hardware, including an OCT scanner, or a mix of dedicated hardware and software instructions, stored on a non-transitory computer readable medium and executed by an associated processor. Further, it will be appreciated that the term “axially,” as used here, refers to an axis substantially parallel to a direction of emission of light from the OCT scanner. At 102, an optical coherence tomography scan of the region of interest is performed to produce at least one set of A-scan data. At 104, an axial location of the surgical instrument is identified from the at least one set of A-scan data. At 106, an axial location of the target, such as a tissue structure or surface, is identified from the at least one set of A-scan data. It will be appreciated that the determination of the axial locations in 104 and 106 can be determined by an appropriate pattern recognition algorithm.

At 108, a relative distance between the surgical instrument and the target is calculated. At 110, the calculated relative distance between the surgical instrument and the target is communicated to the surgeon in real time via one of a visual and an auditory feedback element. For example, the feedback can include a numerical or graphical representation on an associated display, or a change in an audible or visual indicator responsive to the calculated relative distance. It will be appreciated that some delay will be necessary to process the OCT scan data, and “real time” is used herein to indicate that the processing represented by 104, 106, 108, and 110 is performed in a sufficiently small interval such that a change in the calculated relative distance is communicated to the surgeon in a manner that a human being would perceive as immediately responsive to a movement of the instrument. Accordingly, the relative position communicated to the surgeon can be directly utilized in the performance an OCT-guided surgical procedure.

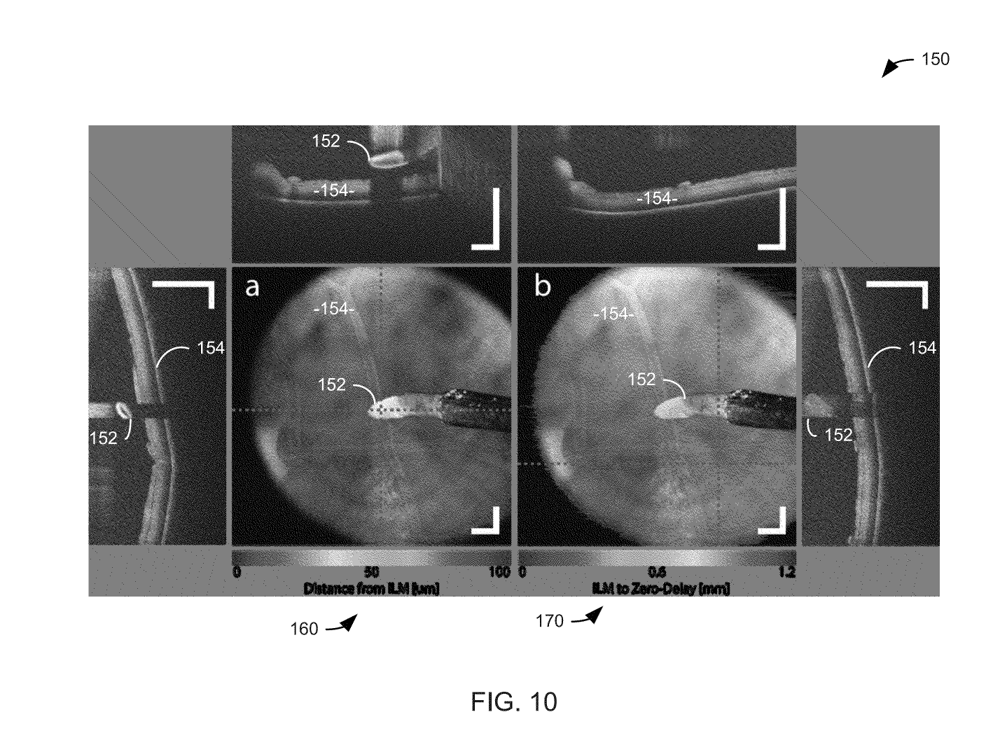

FIG. 10 illustrates one example of an OCT dataset 150 comprising multiple views of an ophthalmic scraper 152 above the retina 154. In one example, the image of FIG. 10 could be the feedback provided to the user or the part of the analysis system that is used to compute relative distance by the feedback element 22. Here, both the surface of the retina 154, specifically the internal limiting membrane (ILM) and the instrument 152 were segmented and each of the distance between the instrument and tissue surface 160 and distance between the tissue surface and a zero-delay representation of the OCT 170 are overlaid onto of a structural OCT en face view as colormaps. In this example, visual feedback is used to guide surgical maneuvers by relaying precise axial positions of the instrument 152 relative to the tissue layer of interest 154. This can be extended to guide maneuvers on various specific tissue layers and multiple instruments. Similarly, different feedback mechanisms in addition to visual may be employed, including audio and tactile feedback to the surgeon.

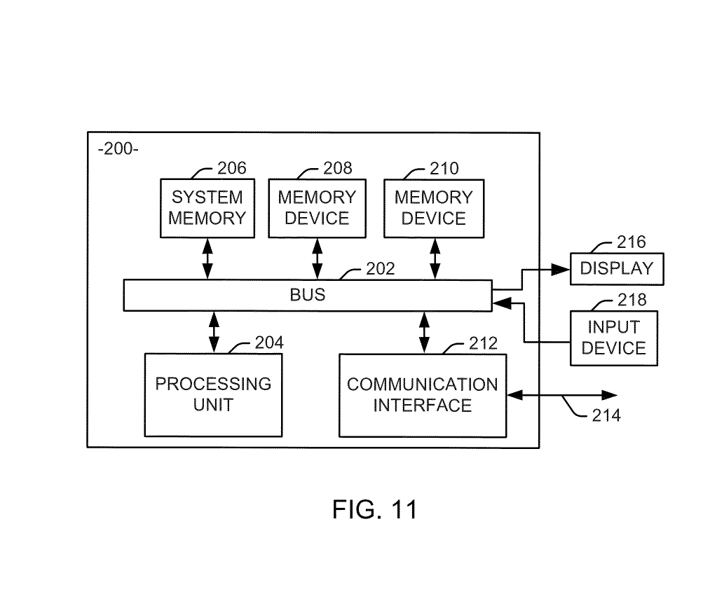

FIG. 11 is a schematic block diagram illustrating an exemplary system 200 of hardware components capable of implementing examples of the systems and methods disclosed herein, such as the instrument tracking system described previously. The system 200 can include various systems and subsystems. The system 200 can be a personal computer, a laptop computer, a workstation, a computer system, an appliance, an application-specific integrated circuit (ASIC), a server, a server blade center, a server farm, etc.

The system 200 can include a system bus 202, a processing unit 204, a system memory 206, memory devices 208 and 210, a communication interface 212 (e.g., a network interface), a communication link 214, a display 216 (e.g., a video screen), and an input device 218 (e.g., a keyboard, touch screen, and/or a mouse). The system bus 202 can be in communication with the processing unit 204 and the system memory 206. The additional memory devices 208 and 210, such as a hard disk drive, server, stand-alone database, or other non-volatile memory, can also be in communication with the system bus 202. The system bus 202 interconnects the processing unit 204, the memory devices 206-210, the communication interface 212, the display 216, and the input device 218. In some examples, the system bus 202 also interconnects an additional port (not shown), such as a universal serial bus (USB) port.

The processing unit 204 can be a computing device and can include an application-specific integrated circuit (ASIC). The processing unit 204 executes a set of instructions to implement the operations of examples disclosed herein. The processing unit can include a processing core.

The additional memory devices 206, 208 and 210 can store data, programs, instructions, database queries in text or compiled form, and any other information that can be needed to operate a computer. The memories 206, 208 and 210 can be implemented as computer-readable media (integrated or removable) such as a memory card, disk drive, compact disk (CD), or server accessible over a network. In certain examples, the memories 206, 208 and 210 can comprise text, images, video, and/or audio, portions of which can be available in formats comprehensible to human beings.

Additionally or alternatively, the system 200 can access an external data source or query source through the communication interface 212, which can communicate with the system bus 202 and the communication link 214.

In operation, the system 200 can be used to implement one or more parts of an instrument tracking system in accordance with the present invention. Computer executable logic for implementing the composite applications testing system resides on one or more of the system memory 206, and the memory devices 208, 210 in accordance with certain examples. The processing unit 204 executes one or more computer executable instructions originating from the system memory 206 and the memory devices 208 and 210. The term “computer readable medium” as used herein refers to a medium that participates in providing instructions to the processing unit 204 for execution.

From the above description of the invention, those skilled in the art will perceive improvements, changes, and modifications. Such improvements, changes, and modifications within the skill of the art are intended to be covered by the appended claims.

Having described the invention, we claim:

1. A system for tracking a depth of a surgical instrument in an optical coherence tomography (OCT) guided surgical procedure comprising:
an OCT device configured to image a region of interest to provide OCT data;
a scan processor configured to determine a relative position of the instrument and a target within the region of interest from at least the OCT data, where the instrument is one of in front of the target, within the target, or below the target; and
a feedback element configured to communicate the relative position of the instrument and the target to a user in a human comprehensible form.

2. The system of claim 1, wherein the region of interest is a point on the target, and the OCT device is configured to provide an axially aligned A-scan, the scan processor being configured to determine a relative position of the instrument and a target from the axially aligned A-scan within the axis defined by the A-scan.

3. The system of claim 2, wherein the axially aligned A-scan is a first axially aligned A-scan, and the OCT device is configured to determine a position of the instrument from the first axially aligned A-scan and a position of the target from the second axially aligned A-scan.

4. The system of claim 1, wherein the region of interest is a plane including at least a portion of the target, and the OCT device is configured to provide a cross-sectional B-scan, the scan processor being configured to determine a relative position of the instrument and a target from the cross-sectional B-scan.

5. The system of claim 4, the scan processor comprising a pattern recognition classifier configured to identify at least one of the instrument and the target with the cross-sectional B-scan.

6. The system of claim 5, wherein the pattern recognition system utilizes a template matching algorithm to match a portion of the cross-sectional B-scan to one of a plurality of templates representing the instrument.

7. The system of claim 1, wherein the feedback element communicates the relative position of the instrument and the target to a user as an audible signal.

8. The system of claim 1, wherein the feedback element communicates the relative position of the instrument and the target to a user through a visual feedback element incorporating a structural OCT en face view, the visual feedback representing a distance between the target and the instrument in a zero-delay representation of the OCT data.

9. The system of claim 1, wherein the feedback element communicates the relative position of the instrument and the target to a user as a numerical indicator.

10. The system of claim 1, wherein the feedback element communicates the relative position of the instrument and the target to a user as tactile feedback.

11. The system of claim 1, wherein the system further comprises a sensor for detecting spectroscopic scattering from the instrument.

12. The system of claim 11, wherein the system further comprises a radiation source attached to the instrument, the radiation source being configured to provide electromagnetic radiation at a wavelength associated with the sensor.

13. The system of claim 1, wherein the system further comprises an optical marker attached to the instrument, the scan prcoessor being configured to locate the optical marker in the OCT data.

14. The system of claim 1, wherein each of the scan processor and the feedback element are configured to provide the relative position of the instrument and the target to a user in real time, such that a change in the calculated relative distance is communicated to the user after a sufficiently small interval as to be perceived as immediately responsive to a movement of the instrument.

15. A computer-implemented method for communicating a relative location of a surgical instrument and a target within a region of interest to a surgeon comprising:
performing an optical coherence tomography scan of the region of interest to produce at least one A-scan;
identifying an axial location of the surgical instrument from the at least one A-scan;
identifying an axial location of the target from the at least one A-scan;
calculating a relative distance between the surgical instrument and the target; and
communicating the calculated relative distance between the surgical instrument and the target to the surgeon via one of a visual, a tactile, and an auditory feedback element;
wherein each of identifying the axial location of the surgical instrument, identifying the axial location of the target, calculating the relative distance, and communicating the calculated relative distance are performed in real time, such that a change in the calculated relative distance is communicated to the surgeon after a sufficiently small interval as to be perceived as immediately responsive to a movement of the instrument.

16. The method of claim 15, wherein performing the optical coherence tomography scan of the region of interest to produce the at least one A-scan comprises performing the optical coherence tomography scan of the region of interest to produce first and second A-scans, identifying the axial location of the surgical instrument comprises identifying the axial location of the surgical instrument from the first A-scan, and identifying the axial location of the target comprises identifying the axial location of the target from the second A-scan.

17. The method of claim 15, wherein performing the optical coherence tomography scan of the region of interest to produce the at least one A-scan comprises performing an optical coherence tomography scan of the region of interest to produce a plurality of A-scans and combining them to provide a B-scan, and identifying the axial location of the surgical instrument and identifying the axial location of the target comprises identifying the axial locations of the surgical instrument and the target from the B-scan.

18. The method of claim 17, wherein identifying an axial location of the surgical instrument from the B-scan comprises identifying the surgical instrument via a pattern recognition algorithm.

19. A system for tracking a surgical instrument in an optical coherence tomography (OCT) guided, ophthalmic surgical procedure comprising:
an OCT device configured to image a region of interest to provide OCT data;
a scan processor configured to determine an axial position of the surgical instrument and an axial position of a target within the region of interest from the OCT data, the scan processor comprising a pattern recognition classifier to identify at least one of the instrument and the target; and
a feedback element configured to communicate at least a relative position of the instrument and the target to a user in a human comprehensible form.

20. The system of claim 19, wherein each of the scan processor and the feedback element are configured to provide the relative position of the instrument and the target to a user in real time, such that a change in the calculated relative distance is communicated to the user after a sufficiently small interval as to be perceived as immediately responsive to a movement of the instrument.

21. The system of claim 19, wherein the pattern recognition algorithm includes a template matching algorithm configured to match a portion of the OCT data to one of a plurality of templates representing the instrument.
